(19) United States
(12) Patent Application Publication
(10) Pub. No.: US 2023/0288297 A1
(43) Pub. Date: Sep. 14, 2023

(54) SAMPLE COLLECTION DEVICE AND METHOD FOR EXTRACTING PARTICLES FROM A FLUID SAMPLE

(71) Applicant: Yair TAL, Rehovot (IL)

(72) Inventor: Yair TAL, Rehovot (IL)

(21) Appl. No.: 18/018,355

(22) PCT Filed: Jul. 29, 2021
(86) PCT No.: PCT/IL2021/050919

§ 371 Date: Jan. 27, 2023

(30) Foreign Application Priority Data
Jul. 29, 2020 (IL) 276367

(51) Int. Cl. G01N 1/22; B01D 46/00; G01N 15/06; G01N 33/497

(52) U.S. Cl. CPC G01N 1/2205; B01D 46/0028; G01N 15/0618; G01N 33/497; G01N 2001/4088

(57) ABSTRACT

The present disclosure discloses device and method for extracting a sample of particles, from a fluid sample, such that the sample of particles having a volumetric concentration sufficient for further analysis. This technique is advantageous when the original fluid sample having a volumetric concentration of particles under the threshold for their analysis.

### TECHNOLOGICAL FIELD

The present disclosure is in the field of collecting fluid samples to test and analyze particles that are present within the fluid sample.

### BACKGROUND ART

References considered to be relevant as background to the presently disclosed subject matter are listed below:
WO 2017/117545
CN 204330430
U.S. Pat. No. 7,964,389

Acknowledgement of the above references herein is not to be inferred as meaning that these are in any way relevant to the patentability of the presently disclosed subject matter.

### GENERAL DESCRIPTION

The present disclosure discloses device and method for extracting a sample of particles, from a fluid sample, such that the sample of particles having a volumetric concentration sufficient for further analysis. This technique is advantageous when the original fluid sample having a volumetric concentration of particles under the threshold for their analysis.

The terms “particles” and “particles of interest” throughout the application refer to non-electrical charged, electrical charged, non-magnetic or magnetic non-organic particles or organic particles including volatile organic compounds (VOCs), non-living or living particles such as airborne microbial agents or microorganisms such as viruses, or other contaminations.

The term “fluid” throughout the application refers to any of gas or liquid, for example the fluid may be gas collected from the environment or chimney or air exhaled by a subject or water sampled from a water container.

The term “analysis” throughout the application refers to any observation, identification, post studies or tests of the sample to derive characteristics of the particles in the sample, identifying their presence, their quantity and/or concentration.

The term “volumetric concentration” throughout the application refers to the quantity of particles per a volume unit.

The term “analysis threshold” throughout the application refers to the minimum of the volumetric concentration level of the particles required for the particles' analysis.

The device includes a fluid inlet for introduction of fluid sample containing particles of interest, e.g. air exhale of a subject into the device, liquid of a water source such as a spring or contaminated gas such as gas from a chimney. The fluid is introduced into the device to flow through a sampling tube from its first end to its second end. The amount of fluid, namely the volume amount that flows through the device is measurable by a volume indicator located at the flow path of the fluid sample in the device. The volume indicator is configured to give an indication about the volume of the fluid that flows through the device, e.g. a flowmeter, a reservoir with tick marks, deformable reservoir with identifiable volumes states or any other means that can gives indication for the volume of fluid sample is introduced into the device. A filter element configured for blocking the flow of the particles along the device to trap them in a trapping zone to thereby extracting the particles from the original fluid sample. The filter may be a porous membrane allowing passing of fluids and blocking the passing of the particles or any other filter that traps the particles of interest in the trapping zone. The trapping zone is defined by a fluid-tight sealed volume upstream the filter such that at least part of the trapping zone is a volume within the sampling tube. After the sampling collection is done, the trapping zone includes a particle sample that has a volumetric concentration of the particles above that of the original fluid sample. The objective is to get a volumetric concentration of the particles above their analysis threshold, and to have indication of their volumetric concentration in the original fluid sample. The increase factor between the volumetric concentration of the extracted particles in the trapping zone and the volumetric concentration of the particles in the original fluid sample is equal to the ratio between the volume of the fluid passing through the sampling tube to the volume of the trapping zone. Since the trapping zone volume is a known parameter and the volume of introduced fluid sample is measurable, the increase factor can be easily derived.

In the embodiment where the volume indicator is a deformable reservoir, there can be two alternatives configurations of locating the deformable reservoir with respect to the sampling tube—upstream or downstream. It is to be noted that the deformable reservoir can be a bag, e.g. a plastic or paper bag or any reservoir that is capable of deformation or any reservoir that is capable of deformation of at least a portion of its walls or by being shaped for allowing it such as a bellow shape or by including a piston.

In the first configuration, the reservoir is located upstream the sampling tube along the flow path of the sampled fluid such that fluid introduced through the inlet is first being received in the reservoir through a reservoir inlet and accumulated therein. After sufficient fluid is accumulated in the deformable reservoir, it is controllably discharged, e.g. by applying a force on the reservoir that forces the fluid to flow through a reservoir outlet into the sampling tube and then out of the device. During the flow of the fluid along the sampling tube, the particles in the fluid are blocked by the filter and are trapped in the trapping zone.

In a second configuration, a reservoir, either a deformable reservoir or a reservoir with identifiable marks, is located downstream the sampling tube and is linked to the second end of sampling tube, i.e. to its outlet portion, and the fluid flows towards the reservoir to fill it with fluid up to the desired amount that is indicated by noticeable accumulation of the fluid within the reservoir up to a certain volume. During the flow of the fluid towards the reservoir, the particles in the fluid are blocked by the filter and are trapped in the trapping zone.

In an optional design of the second configuration, a unidirectional valve is disposed at or between the fluid inlet and the filter. Fluid passing through the unidirectional valve flows towards the filter and the particles in the fluid sample are blocked by the filter to be trapped within the trapping zone. Therefore, the trapping zone is defined by the volume confined between the unidirectional valve and the filter. Optionally, in this configuration, the first end of the sampling tube constitutes the fluid inlet.

Thus, a first aspect of the present disclosure provides a sample collection device for extracting particles from a sample of fluid, e.g. organic, non-organic, volatile organic compounds (VOCs), non-living or living particles such as airborne microbial agents from an air sample of a subject. The device includes a fluid inlet that is configured to receive a fluid therethrough. The fluid may be any of gas or liquid, for example the gas may be air exhaled by a subject, contaminating gas or liquid of a potable water source.

The device further includes a sampling tube with first and second open ends. The first end is upstream the second end with respect to flow path of the sample of fluid through the device, namely the first end being more proximal to the fluid inlet than the second end. The sampling tube includes a filter located between the first and second ends for blocking passage of the particles upon a flow of the fluid therethrough to thereby trapping said particles in a trapping zone defined upstream the filter.

In some embodiments of the device, the volume indicator is a deformable reservoir.

In some embodiments, the deformable reservoir is linked to the first or second ends of the sampling tube. The deformable reservoir can be configured for undergoing deformation upon receiving fluid being introduced through said inlet and to increase its volume from at least a first volume to a second volume. Alternatively, when the deformable reservoir contains fluid sample, it can be pressed by outer force or pressure and be deformed and decreased its volume from a first volume to a second volume, and forces egress flow of the fluid sample from its interior through a deformable reservoir outlet towards the sampling tube, when the deformable reservoir is linked to the first end. It is to be noted that the first volume may be either greater or smaller than the second volume and that the reservoir may undergo reversible transition between its different volume states. The first and second volumes of the deformable reservoir are identifiable such that the user of the device can recognize that sufficient fluid is introduced or discharged from the reservoir.

In the embodiments that the deformable reservoir is fluid-tight coupled to the first end of the sampling tube, the deformable reservoir may be indirectly coupled to the fluid inlet, namely such that fluids that are introduced through the inlet may flow through components of the device before being received in the deformable reservoir. At the first stage the deformable reservoir is being filled with a fluid sample to a desired volume. Later, upon force or pressure pressing on at least a portion of its walls, the volume of the reservoir is decreased from the first volume to the second volume, which is smaller than the first volume, and the fluid is forced to flow into the sampling tube. The volume difference between the first volume and the second volume are indicative by definite forms of the deformable reservoir for the first and second volumes, marks, or pattern of the deformable reservoir.

In the embodiments that the deformable reservoir is located downstream the sampling tube and is linked to the second end, namely, the fluid flows from the fluid inlet, through the sampling tube and into the deformable reservoir, results in an increase of the volume of the reservoir. Marks of the reservoir give indication for the volume of the fluid accumulated in the reservoir. For example, a pattern on the walls of the deformable reservoir can give the desired indication that sufficient volume of fluid is accumulated therein.

In some embodiments, the device further includes a sealing member configured to allow fluid-tight sealing of the volume of the trapping zone, upon the end of the sampling process, namely when sufficient volume of fluid passed through the sampling tube. Therefore, the sealing member can be in two different states, an open state wherein fluid flows into the trapping zone and a sealing state wherein the volume of the trapping zone is sealed.

In some embodiments of the device, the sealing member is configured to seal the first end of the sampling tube.

In some embodiments, the device includes a unidirectional valve that is located at the flow path between the fluid inlet and the filter for allowing introduction of fluid sample into the sampling tube and blocking discharge of fluid sample from the sampling tube towards or via the fluid inlet. In some embodiments, the unidirectional valve is located between the first end and the filter.

In some embodiments of the device, the first end is fluid-tight coupled to the deformable reservoir and the fluid inlet is formed in or disposed upstream the deformable reservoir. The deformable reservoir comprises a deformable reservoir inlet for receiving fluid being introduced via said deformable reservoir inlet. The deformable reservoir fluid inlet is sealable upon filling said deformable reservoir with a fluid to said first volume, and upon connecting the deformable reservoir fluid inlet to the first end and releasing the fluid sample from the reservoir towards the sampling tube, e.g. by applying an external force or pressure on at least a portion of the deformable reservoir walls, the volume of the deformable reservoir decreases to the second volume, and the fluid sample is forced to flow through said sampling tube, thereby causing or forcing said particles for being trapped in said trapping zone.

In some embodiments of the device, the deformable reservoir, in its second volume, is configured to fit into the tube and the sealing member is configured to seal the first end when the deformable reservoir is in the tube.

In some embodiments of the device, the deformable reservoir comprises elastic walls and the deformable reservoir is configured for changing its volume upon a change of the pressure in its interior, namely upon change of forces applied onto its walls. Thus, when the internal pressure of the reservoir increases, so its volume increases and when the internal pressure decreases, its volume decreases. For example, when the deformable reservoir is filled with fluid and the pressure in its interior is above the ambient, the flow of fluid from the reservoir downstream along the flow path is spontaneous. It is to be noted that the deformable reservoir may also undergo, at least in part of its deformation, isobaric deformation, namely a change of volume without the change of its internal pressure.

In some embodiments, the device includes a visual indicator for indicating of sufficient fluid volume that is introduced into the deformable reservoir.

In some embodiments of the device, the indicator is a pattern on the deformable reservoir that is identifiable upon inflation of the deformable reservoir to a sufficient volume.

In some embodiments of the device, the deformable reservoir is configured for changing its volume upon a force acts on at least portion of its walls.

In some embodiments of the device, at least a portion of the walls of the deformable reservoir is elastic.

In some embodiments of the device, wherein at least a portion of the deformable reservoir walls is in the form of a bellows.

In some embodiments of the device, the reservoir has flexible walls for allowing its deformation. It is to be noted that the flexible walls may constitute only a portion of the entire walls of the reservoir.

In some embodiments of the device, the reservoir is inflatable and compressible.

In some embodiments of the device, the deformable reservoir includes a piston.

In some embodiments of the device, the tube is coupled to the deformable reservoir in an attachable/detachable manner for allowing the extraction of the tube from the entire device to facilitate quantification tests of the sample.

In some embodiments of the device, the volume indicator is a non-deformable reservoir linked to the second end of the sampling tube. The reservoir comprises marks for indicating the amount of fluid that is introduced therein.

In some embodiments of the device, the non-deformable reservoir comprises aperture for allowing release of gas from its interior.

In some embodiments of the device, the volume indicator is a flowmeter.

In some embodiments of the device, the flowmeter is located at the flow path between the first and second ends of the sampling tube.

In some embodiments of the device, the flowmeter is located before the sampling tube, namely upstream the first end.

In some embodiments of the device, the flowmeter is located after the sampling tube, namely downstream the second end.

In some embodiments of the device further includes a closure configured for association with the second end in two states: a first state for fluidically sealing the second end and a second state for allowing flow of fluid through the second end.

In some embodiments the volume indicator is constituted by a combination of the flowmeter and the deformable reservoir that are described above.

In some embodiments of the device, the fluid is gas or liquid. In some specific embodiments, the fluid is air exhaled from a subject.

In some embodiments of the device, the inlet is a mouthpiece to allow a subject to exhale air through the fluid inlet into the device.

In some embodiments of the device, the inlet is sealable.

In some embodiments of the device, at least a portion of the trapping zone walls is made of transparent material for allowing visual inspection of its interior.

In some embodiments, the particles are at least one of the following: non-organic, non-electrical charged, electrical charged, nonmagnetic, magnetic, volatile organic compounds (VOCs), non-living particle, and living particles such as microbial agents and viruses.

In some embodiments of the device, the filter is a microbial filter selected from bacterial and viral filter. In some embodiments, said viral filter is configured for filtering out SARS-COV-2 viruses.

In some embodiments of the device, the filter is configured to filter-out particles having a diameter of at least 75 nm, 90 nm or 100 nm and larger.

In some embodiments of the device, the filter is realized by field of force induced in the said sampling tube configured for trapping the said particles in the trapping zone. In some embodiments, the field force is electromagnetic field. In some other embodiments, the field force is acceleration field.

Another aspect of the present disclosure provides a method for extracting particles from a fluid sample, e.g. gas or liquid sample, for further analysis thereof. For example, the gas sample may be an exhale air sample of a subject, and the liquid may be water or any other liquid of interest. The method includes (a) introducing a volume of fluid sample into a sample collection device through a fluid inlet. The sample collection device comprises a sampling tube with first and second open ends, the first end being more proximal to the fluid inlet than the second end. The tube comprises a filter disposed between the first and second ends for blocking passage of said particles upon a flow of the fluid sample therethrough to thereby trapping said particles in a trapping zone defined upstream said filter.

The method further includes (b) passing the fluid sample from the first end to the second end thereby trapping the particles in said trapping zone.

The method further includes (c) passing the fluid sample through a volume indicator or receiving the fluid sample in a volume indicator, said volume indicator is configured to provide indication of the volume of the fluid sample passing through said sampling tube.

In some embodiments of the method, (c) is carried out prior to (b), in some embodiments (c) is carried out after (b) and in some other embodiments, (c) is carried out simultaneously with (b).

In some embodiments, the volume indicator is a flowmeter disposed along the flow path of the fluid sample in the sample collection device.

In some embodiments of the method, the volume indicator is a deformable reservoir capable of changing its volume between at least a first and second volumes, and wherein one of the first and second ends are fluid-tight coupled to the deformable reservoir such that upon a flow of the fluid that causes or resulting in said change of volume of the deformable reservoir, said particles are being trapped in said trapping zone.

In some embodiments of the method, the sample collection device comprises a sealing member and the method further comprising fluid-tight sealing the volume of the trapping zone.

In some embodiments of the method, the sealing member is configured to seal the first end of the sampling tube.

In some embodiments of the method, the sample collection device comprises a unidirectional valve disposed at a flow path between said fluid inlet and said filter for allowing introduction of fluid sample into the sampling tube and blocking discharge of fluid sample from the sampling tube towards or via the fluid inlet.

In some embodiments of the method, the volume indicator is a deformable reservoir capable of changing its volume between at least a first and second volumes, and wherein one of the first and second ends are fluid-tight coupled to the deformable reservoir such that upon a flow of the fluid that causes or resulting in said change of volume of the deformable reservoir, said particles are being trapped in said trapping zone.

In some embodiments of the method, the deformable reservoir is fluid-tight coupled to the second end and the fluid inlet disposed at or upstream said first end of the sampling tube, said second volume being larger than the first volume.

In some embodiments of the method, the first end being fluid-tight coupled to the deformable reservoir and the fluid inlet is formed in or disposed upstream the deformable reservoir, the device further includes a closure configured for association with the second end in two states: a first state for fluidically sealing the second and a second state for allowing flow of fluid through the second end. The deformable reservoir comprises a deformable reservoir inlet for receiving fluid being introduced via said fluid inlet when the closure is at the first state, according to (a). The method further comprising sealing the fluid inlet after (a), switching the closure to the second state and thereafter pressing the deformable reservoir to carry out (b) and (c).

In some embodiments of the method, the volume indicator is a deformable reservoir capable of changing its volume between at least a first and second volumes. The deformable reservoir comprises a deformable reservoir inlet for receiving fluid being introduced via said fluid inlet, according to (a). The method further comprising sealing said deformable reservoir inlet after filling the deformable reservoir with fluid sample to said first volume, fluidically connecting the deformable reservoir inlet to the first end and releasing the fluid sample from the reservoir towards the sampling tube to decrease the volume of the deformable reservoir to the second volume, resulting in a flow of fluid through said sampling tube, thereby causing or forcing said particles for being trapped in said trapping zone.

In some embodiments, the method further comprising introducing the deformable reservoir into the tube and fluid-tight sealing the first end of the sampling tube thereafter with a sealing member.

In some embodiments of the method, the deformable reservoir comprises walls that at least portion thereof is flexible or deformable.

In some embodiments of the method, the inlet is a mouthpiece.

In some embodiments of the method, the reservoir is inflatable and/or compressible.

In some embodiments of the method, the reservoir includes a piston.

In some embodiments of the method, the filter is a microbial filter selected from bacterial and viral filter. In some embodiments, the filter is configured to filter-out particles having a diameter of at least 75 nm, 90 nm or 100 nm, such as SARS-COV-2 viruses.

In some embodiments, the method further includes sealing the second end of the sampling tube.

In some embodiments of the method, the sample collection device is any one of the above described embodiments, or any combination thereof.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1F are schematic illustrations of a longitudinal cross-sections of a non-limiting example of an embodiment of the device of the present disclosure, presenting different phases of the device during a sampling process. In this example, the deformable reservoir is expanded during the fluid collection and compressed during the sampling process.

FIGS. 2A-2E are schematic illustrations of a longitudinal cross-sections of another non-limiting example of an embodiment of the device of the present disclosure, presenting different phases of the device during a sampling process. In this example, the deformable reservoir is compressed during the sampling process.

FIGS. 3A-3F are schematic illustrations of a longitudinal cross-sections of another non-limiting example of an embodiment of the device of the present disclosure, presenting different phases of the device during a sampling process. In this example, the deformable reservoir is expanded during the fluid collection separately from the sampling tube and compressed during the sampling process where it is linked to the sampling tube.

FIG. 4 is a schematic illustration of a cross-sectional view of another non-limiting example of an embodiment of the device according to an aspect of the present disclosure presenting the basic device with a unidirectional valve and filter with a flowmeter that is applied along the flow path of the fluid sample.

FIG. 5 is a schematic illustration of a cross-sectional view of another non-limiting example of an embodiment of the device according to an aspect of the present disclosure showing a device with an unidirectional valve and filter and with a deformable reservoir as a volume indicator.

FIG. 6 is a schematic illustration of a cross-sectional view of another non-limiting example of an embodiment of the device according to an aspect of the present disclosure presenting the basic device to trap particles in a trapping zone confined by a filter and unidirectional valve.

FIG. 7 is a schematic illustration of a cross-sectional view of another non-limiting example of an embodiment of the device according to an aspect of the present disclosure showing a device with a non-deformable reservoir as a volume indicator, having marks on it indicating the volume of fluid that is introduced thereinto.

### DETAILED DESCRIPTION OF EMBODIMENTS

The present disclosure provides a sample collection device that is configured to receive a large volume of a fluid sample containing particles of interest, e.g. microbes, viruses, pathogens, contaminations, etc., and extract therefrom a concentrated portion having a volumetric concentration of particles above the analysis threshold. The concentrated portion is a smaller volume of fluid than the entire fluid sample and contains higher volumetric concentration of particles of interest than originally found in the fluid sample. The volume of the concentrated portion vs the volume of the entire fluid sample gives the increase factor and serves to derive the original concentration of the particles in the original sample. For example, if the sample is received from an exhale of a subject, the device output of the sample is the volume of the trapping zone that contains a concentrated sample volume that is smaller than the entire air volume that the subject introduced into the device. By trapping the particles of interest in the trapping zone, e.g. by using a selective filter for filtering the desired particles (for example, according to their size), this concentrated portion includes higher density of volumetric concentration than the original sample volume, which facilitates to apply analysis on the concentrated portion. The exhaled air volume into the device may be, for example, 1 liter and the volume of the trapping zone may be 1 ml such that the increase factor is 1000. This means that the output of the sample collection device yields a particle volumetric concentration 1000 folds higher than the original fluid sample.

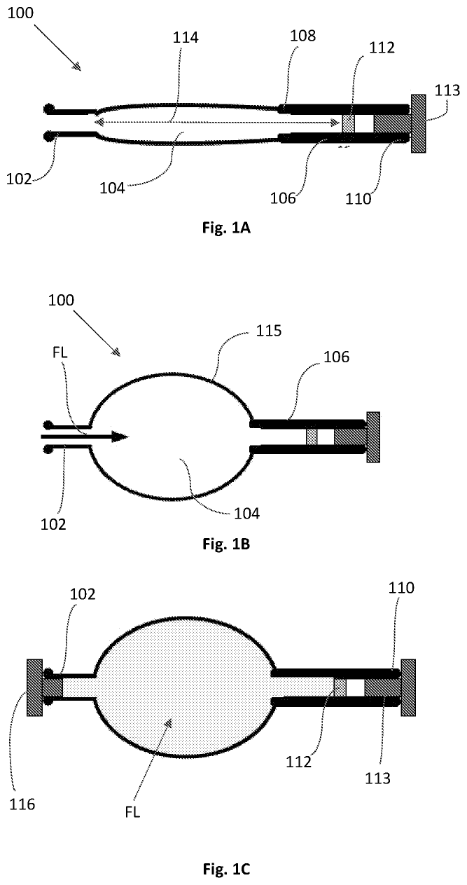

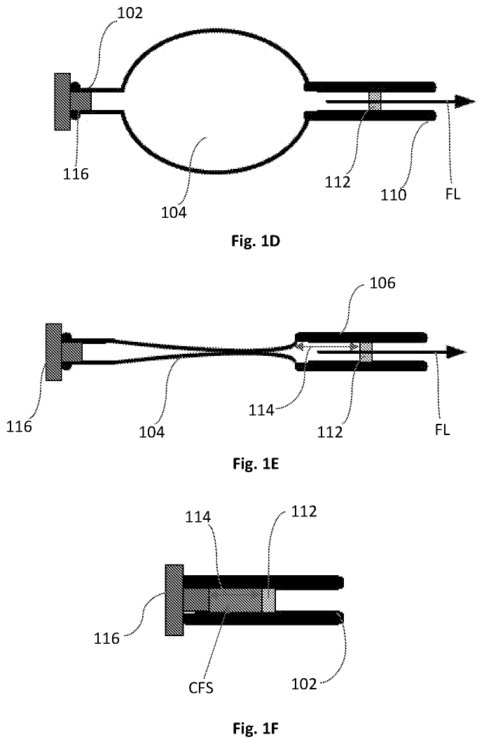

Reference is first made to FIGS. 1A-1F, which are schematic illustration of cross-sectional views of a non-limiting example of a sample collection device according to an embodiment of the present disclosure. Each figure shows the sample collection device at different phase of the sample collection and sample process. FIG. 1A shows a device 100 with a fluid inlet 102, in the form of a mouthpiece, that is linked to a deformable reservoir 104. The deformable reservoir 104 is gas-tight coupled to a sampling tube 106 extending between a first proximal end 108 and a second distal end 110. A particles filter 112 is disposed between the first end 108 and the second end 110 and is configured to filter particles above a certain size from passing therethrough towards the second end 110. The second end 110 is sealed by a removable closure 113. A trapping zone 114 is defined between the fluid inlet 102 and the filter 112 such that substantially any particle above a certain size that is introduced through the inlet 102 is barred from passing through the filter and remains therein, namely in the zone between the inlet 102 and the filter. During the sampling process, the trapping zone 114 may change its size until it reaches the eventual end size at the end of the sampling process. In some embodiments, the trapping zone 114 may be limited to be defined between the first end 108 and the filter 112.

Upon introduction of fluid FL through the inlet 102, the deformable reservoir is inflated, due to its flexible walls 115, as can be seen in FIG. 1B. Thus, in its passive, non-biased state, the deformable reservoir 104 is non-inflated and its volume increases when fluid is introduced thereto. The inflation of the reservoir 104 is made up to a predetermined and measurable volume such that the amount of fluid that is introduced into the device 100 is known for calculation of the increase factor at the output of the device. An inlet sealing member 116 is then fitted to the inlet 102 thereby gas-tight sealing it to trap all the fluid FL within the reservoir 104.

The removable closure 113 that fits in the second end 110 seals the device such that the fluid within the reservoir 104 remains therein as long as the closure remains plugged in the second end 110, as can be seen in FIG. 1C. Upon opening of the closure 113, the fluid within the reservoir drains through the second end 110 and the particles above the certain size are blocked by the filter 112 and remain in the trapping zone 114, as can be seen in FIG. 1D. The deformable reservoir 104 returns to its non-inflated size such that substantially all the fluid is discharged from the reservoir towards the second end 110 and into the ambient environment, as can be seen in FIG. 1E.

The inlet sealing member 116 is configured to fit in the first end 108 together with the deflated reservoir 104 such that the trapping zone 114 is confined between the first end 108 and the filter 112, and more specifically between the inlet sealing member 116 and the filter 112. As can be seen in FIG. 1F, the inlet sealing member 116 seals the first end 108 and the trapping zone 114 confining a concentrated fluid sample CFS.

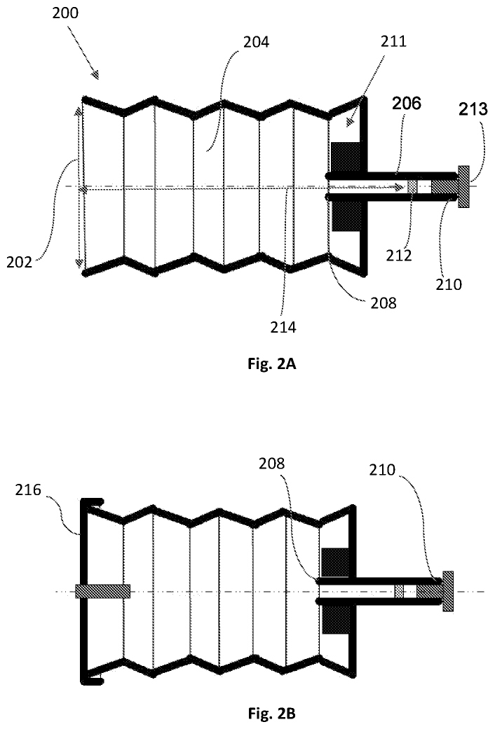

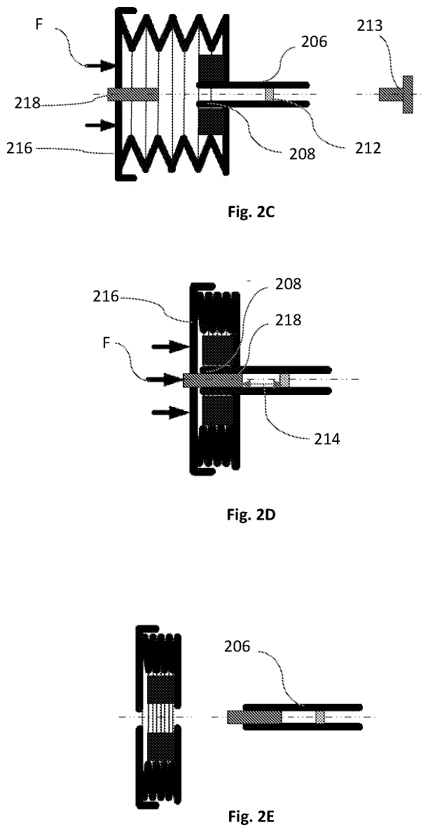

In the figures throughout the application, like elements of different figures were given similar reference numerals shifted by the number of hundreds corresponding to the number of the respective figure. For example, element 202 in FIGS. 2A-2E serves the same function as element 102 in FIGS. 1A-1E.

Another embodiment of the device of the present disclosure is exemplified in FIGS. 2A-2E are schematic illustrations of non-limiting example of another embodiment of a device according to the present disclosure. In this embodiment, the device 200 includes a reservoir 204 that at the onset of the sampling process is at its first, expanded state and defined by an initial volume for receiving fluid sample from the environment through its inlet 202. Once the fluid sample is collected, an inlet sealing member 216 fits over the inlet 202 to confine the fluid sample within the reservoir 204. This can be best seen in FIGS. 2A-2B. A first end 208 of a sampling tube 206 protrudes into a distal end portion 211 of the reservoir 204 such that it is in fluid communication with the reservoir 204. A particles filter 212 configured for blocking passage of particles of interest, e.g. particles above a certain size, is disposed between the first end 208 of the tube 206 and a second, distal end 210 thereof. The second end is fluid-tight sealed by a closure 213 during the collection of the fluid sample to ensure that no fluid flows through the sampling device uncontrollably, thereby allowing to calculate the amount of the fluid sample. A trapping zone 214 for trapping the particles is defined as the confined zone extending between the filter 212 and the sealing member 216. The trapping zone 214 can change its size during the sampling process of the device, such that it includes a portion of the tube and the reservoir at the beginning of the process and at the end it includes merely a portion of the tube, i.e. between the filter and the first end of the tube.

After the inlet sealing member 216 is plugged to the fluid inlet 202, the closure 213 is removed from the second end 210 of the tube 206. By compressing the reservoir 204 with application of force F, the volume of the reservoir decreases to a second volume while fluid drains through the second inlet 210, as can be best seen in FIGS. 2C-2D. The particles of interest in the fluid are blocked by the filter 212 and remain in the trapping zone 214, while the size of it decreases during the compression of the reservoir 204. The inlet sealing member 216 comprises a protrusion that fits into the first end 208 of the tube 206 and serves as a sealing plug 218, thereby sealing it and confining the trapping zone between the filter 212 and the first end 208, or more specifically, between the filter 212 and the plug 218. The plug 218 is decouplable from the inlet sealing member 216 such that after it is plugged into the first end 208 of the tube 206, the tube can be extracted from the reservoir 204 together with the plug 218 to be analyzed.

The rate between the initial volume of the reservoir 204 and the final volume of the trapping zone 214 serves for calculating the increase factor and deriving the real concentration of particles in the original sample.

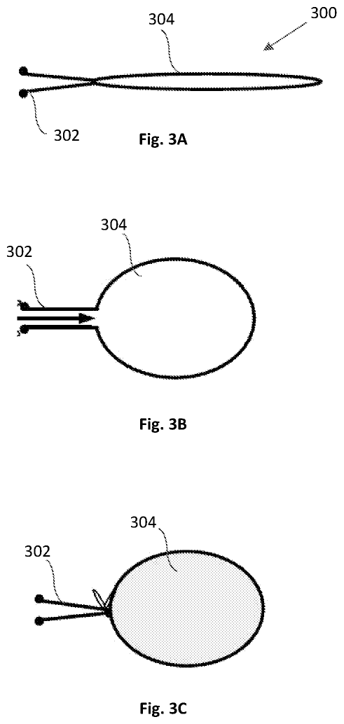

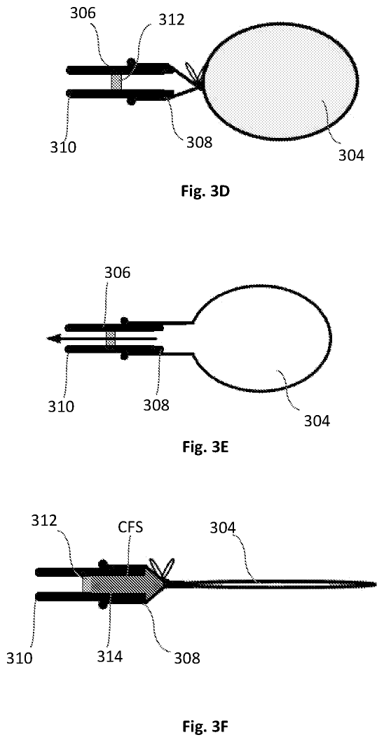

FIGS. 3A-3F are schematic illustrations of yet another non-limiting example of an embodiment of the device of the present disclosure. In this embodiment, the deformable reservoir 304 is filled with the fluid sample while it is separated from the sampling tube 306, as exemplified in FIGS. 3A-3B. Then, the fluid inlet 302 of the reservoir 304 is sealed to confine the fluid sample in the interior of the reservoir, as exemplified in FIG. 3C, and the reservoir is fluid-tight coupled to a first end 308 of the sampling tube 306, as exemplified in FIG. 3D. Once the sealing of the reservoir 304 is removed, the fluid flows from the reservoir 304 through the tube to be discharged through the second, distal end 310 of the tube 306 while passing through the particles filter 312 that is disposed between two ends of the tube 306, as exemplified in FIG. 3E. After all the fluid is drained from the second inlet 310, a concentrated fluid sample CFS remains within the trapping zone 314 that is confined between the filter 312 and the end portion of the reservoir that is coupled to the first end 310 of the tube 306 such that it can be analyzed.

The reservoir 304 is inflated by a desired amount of fluid that can be measurable by a flow meter or by a degree of inflation of the reservoir 304. The rate between the amount of fluid that is introduced into the reservoir and the eventual volume of fluid in the trapping zone 314 serves for calculating the increase factor and deriving the real concentration of particles in the original sample.

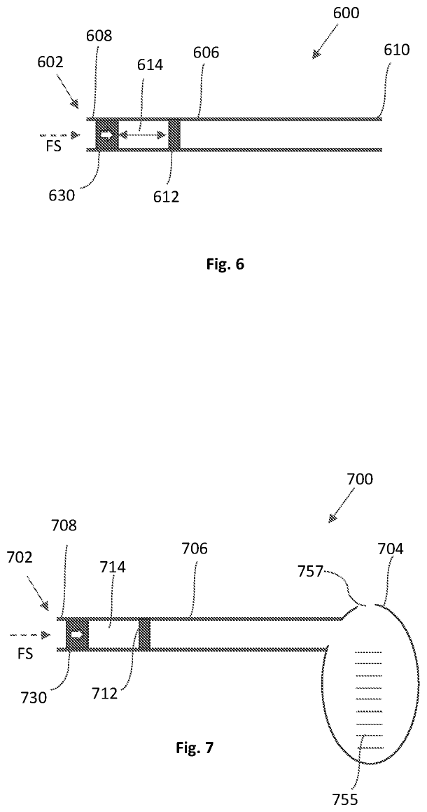

Reference is now made to FIG. 6, which shows a cross-sectional view of an embodiment of the device 600 that includes a sampling tube 606 that is defined between a first end 608 and a second end 610. Fluid sample FS is introduced into the tube 606 via a fluid inlet 602 that is practically constituted by the first end 608 of the tube 606. It is to be noted that the fluid inlet 602 may be constituted by a disposable or non-disposable mouthpiece. The fluid sample FS passes through a unidirectional valve 630 that allows unidirectional flow along the flow path of the tube 606 from the first end 608 towards the second end 610. Fluid sample FS that passes through the unidirectional valve 630 cannot egress back through the fluid inlet 602. A filter 612 is disposed in the tube 606 downstream the unidirectional valve 630 and is configured to block particles of interest that are contained in the fluid sample FS thereby trapping them in a trapping zone 614, which is defined by a volume confined between the unidirectional valve 630 and the filter 614.

The fluid sample FS passes through the tube 632, from the firs end 608 towards the second end 610 and is discharged out of the device through the second end 610 of the tube 606, e.g. to a sterilization device, waste enclosed reservoir, or the environment.

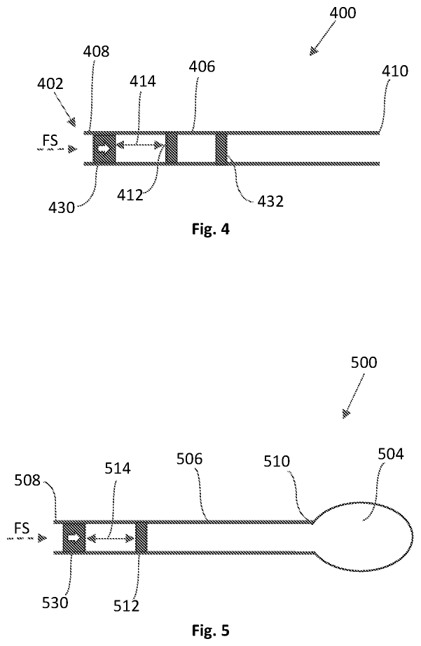

Reference is now made to FIG. 4, which shows a cross-sectional view of an embodiment of the device 400 that includes a sampling tube 406 that is defined between a first end 408 and a second end 410. Fluid sample FS is introduced into the tube 406 via a fluid inlet 402 that is practically constituted by the first end 408 of the tube 406. It is to be noted that the fluid inlet 402 may be constituted by a disposable or non-disposable mouthpiece. The fluid sample FS passes through a unidirectional valve 430 that allows unidirectional flow along the flow path of the tube 406 from the first end 408 towards the second end 410. Fluid sample FS that passes through the unidirectional valve 430 cannot egress back through the fluid inlet 402. A filter 412 is disposed in the tube 406 downstream the unidirectional valve 430 and is configured to block particles of interest that are contained in the fluid sample FS thereby trapping them in a trapping zone 414, which is defined by a volume confined between the unidirectional valve 430 and the filter 414.

A flowmeter 432 is disposed downstream the filter 412 and is configured to measure the amount of fluid (e.g. the volumetric volume) that passes through the tube 406. The flowmeter 432 allows the user to easily determine the amount of total sample fluid that passes through the tube 406 during a sampling session. By measuring the amount of fluid in a sampling session, the original volumetric concentration of the original sample can be derived by determining the volumetric concentration in the trapping zone. It is to be noted that the flowmeter may be disposed at any location along the flow path of the fluid in the tube and its position in FIG. 4 is a mere non-limiting example.

After the fluid sample FS passes through the flowmeter 432, it is discharged through the second end 410 of the tube 406 to the environment.

FIG. 5 is a schematic illustration of a cross-sectional view of an embodiment of the device 500 that includes a sampling tube 506 that is defined between a first end 508 and a second end 510. Fluid sample FS is introduced into the tube 506 via a fluid inlet 502 that is practically constituted by the first end 508 of the tube 506. It is to be noted that the fluid inlet 502 may be constituted by a disposable or non-disposable mouthpiece. The fluid sample FS passes through a unidirectional valve 530 that allows unidirectional flow along the flow path of the tube 506 from the first end 508 towards the second end 510. A filter 512 is disposed in the tube 506 downstream the unidirectional valve 530 and is configured to block particles of interest that are contained in the fluid sample FS thereby trapping them in a trapping zone 514, which is defined by a volume confined between the unidirectional valve 530 and the filter 514. The fluid sample FS that is introduced into the device flows towards a deformable reservoir 504, which is coupled to the second end 510 of the tube 506. The fluid inflates the deformable reservoir 504 to a desired volume, indicating that sufficient fluid sample volume passed through the filter 512 during the sampling session. The particles contained in the sample are trapped in the trapping zone 514 and can be analyzed. By knowing the amount of fluid that is introduced into the deformable reservoir during a sampling session, the amount of the fluid in the trapping zone, and deriving the volumetric concentration in the trapping zone, by any analysis thereof that is usually carried out after the sampling session is over, the original volumetric concentration of the original sample can be derived.

FIG. 7 is a schematic illustration of a cross-sectional view of an embodiment of the device 700 that differs from device 500 of FIG. 5 by including a non-deformable and non-sealed reservoir 704 instead of a deformable reservoir. This embodiment is particularly relevant where the fluid is liquid. The non-deformable reservoir 704 is linked to the sampling tube through its top portion to facilitate liquid flow thereinto. The reservoir 704 includes marks 755 thereon that indicates the amount of fluid filled therein. The reservoir 704 further includes an aperture 757 for allowing release of gas while filling the reservoir with liquid.

1-48. (canceled)

49. A sample collection device for extracting particles from a sample of fluid, comprising:
a fluid inlet;
a sampling tube with first and second open ends, the first end being more proximal to the fluid inlet than the second end, said tube comprises a filter disposed between the first and second ends for blocking passage of said particles upon a flow of the fluid sample therethrough to thereby collecting said particles in a fluid within a trapping volume defined upstream said filter; and
a volume indicator configured to indicate the volume of the fluid sample passing through said sampling tube.

50. The sample collection device of claim 49, comprising a sealing member configured to allow fluid-tight sealing of the volume of the trapping volume;
wherein the sealing member is configured to seal the first end of the sampling tube.

51. The sample collection device of claim 49, comprising a unidirectional valve disposed at a flow path between said fluid inlet and said filter for allowing introduction of fluid sample into the sampling tube and blocking discharge of fluid sample from the sampling tube towards or via the fluid inlet.

52. The sample collection device of claim 49, wherein the volume indicator is a deformable reservoir capable of changing its volume between at least a first and second volumes, and wherein one of the first and second ends are fluid-tight coupled to the deformable reservoir such that upon a flow of the fluid that causes or resulting in said change of volume of the deformable reservoir, said particles are being trapped in said trapping volume.

53. The sample collection device of claim 52, wherein the deformable reservoir is fluid-tight coupled to the second end and the fluid inlet disposed at or upstream said first end of the sampling tube, said second volume being larger than the first volume.

54. The sample collection device of claim 52, wherein the first end being fluid-tight coupled to the deformable reservoir and the fluid inlet is formed in or disposed upstream the deformable reservoir, the device further includes a closure configured for association with the second end in two states: a first state for fluidically sealing the second end and a second state for allowing flow of fluid through the second end,
the deformable reservoir comprises a deformable reservoir inlet for receiving fluid being introduced via said fluid inlet when the closure is at the first state, the fluid inlet either (i) comprises an unidirectional valve or (ii) is sealable by deformation of its structure or by an inlet sealing member upon filling said deformable reservoir with a fluid to said first volume, and upon transition of the closure to the second state, the volume of the deformable reservoir decreases to the second volume, resulting in a flow of fluid through said sampling tube, thereby causing or forcing said particles for being trapped in said trapping volume.

55. The sample collection device of claim 49, wherein the volume indicator is a deformable reservoir capable of changing its volume between at least first and second volumes and the fluid inlet is formed in or disposed upstream the deformable reservoir,
the deformable reservoir comprises a deformable reservoir inlet for receiving fluid being introduced via said deformable reservoir inlet, the deformable reservoir fluid inlet is sealable upon filling said deformable reservoir with a fluid to said first volume, and upon connecting the deformable reservoir fluid inlet to the first end and releasing the fluid sample from the reservoir towards the sampling tube, the volume of the deformable reservoir decreases to the second volume, resulting in a flow of fluid through said sampling tube, thereby causing or forcing said particles for being trapped in said trapping volume.

56. The sample collection device of claim 52, comprising a sealing member configured to allow fluid-tight sealing of the first end of the sampling tube, wherein the deformable reservoir, in its second volume, is configured to fit into the tube and the sealing member is configured to seal the first end when the deformable reservoir is in the tube.

57. The sample collection device of claim 52, wherein at least a portion of the trapping volume walls is made of transparent material for visual inspection of its interior.

58. The sample collection device of claim 49, wherein the volume indicator is a non-deformable reservoir linked to the second end of the sampling tube, said reservoir comprises marks for indicating the amount of fluid that is introduced therein.

59. The sample collection device of claim 52, wherein the tube is coupled to the reservoir in an attachable/detachable manner.

60. The sample collection device of claim 49, wherein the volume indicator is a flowmeter.

61. The sample collection device of claim 49, wherein said filter is a microbial filter selected from bacterial and viral filter.

62. A method for extracting particles from a fluid sample, comprising:
(a) introducing a volume of fluid sample into a sample collection device through a fluid inlet, the sample collection device comprises a sampling tube with first and second open ends, the first end being more proximal to the fluid inlet than the second end, said tube comprises a filter disposed between the first and second ends for blocking passage of said particles upon a flow of the fluid sample therethrough to thereby trapping said particles in a trapping volume defined upstream said filter;
(b) passing the fluid sample from the first end to the second end thereby trapping the particles in said trapping volume;
(c) passing the fluid sample through a volume indicator or receiving the fluid sample in a volume indicator, said volume indicator is configured to provide indication of the volume of the fluid sample passing through said sampling tube.

63. The method of claim 62, wherein (c) is carried out prior to (b).

64. The method of claim 62, wherein the volume indicator is a deformable reservoir capable of changing its volume between at least a first and second volumes, and wherein one of the first and second ends are fluid-tight coupled to the deformable reservoir such that upon a flow of the fluid that causes or resulting in said change of volume of the deformable reservoir, said particles are being trapped in said trapping volume.

65. The method of claim 64, wherein the first end being fluid-tight coupled to the deformable reservoir and the fluid inlet is formed in or disposed upstream the deformable reservoir, the device further includes a closure configured for association with the second end in two states: a first state for fluidically sealing the second and a second state for allowing flow of fluid through the second end,
the deformable reservoir comprises a deformable reservoir inlet for receiving fluid being introduced via said fluid inlet when the closure is at the first state, according to (a),
the method further comprising sealing the fluid inlet after (a), switching the closure to the second state and thereafter pressing the deformable reservoir to carry out (b) and (c).

66. The method of claim 64, wherein the volume indicator is a deformable reservoir capable of changing its volume between at least a first and second volumes,
the deformable reservoir comprises a deformable reservoir inlet for receiving fluid being introduced via said fluid inlet, according to (a),
the method further comprising
sealing said deformable reservoir inlet after filling the deformable reservoir with fluid sample to said first volume,
fluidically connecting the deformable reservoir inlet to the first end and releasing the fluid sample from the reservoir towards the sampling tube to decrease the volume of the deformable reservoir to the second volume, resulting in a flow of fluid through said sampling tube, thereby causing or forcing said particles for being trapped in said trapping volume.

67. The method of claim 64, further comprising introducing the deformable reservoir into the tube and fluid-tight sealing the first end of the sampling tube thereafter with a sealing member.

68. The method of claim 62, wherein the sample collection device is of claim 1.
